(19) United States
(12) Patent Application Publication
(10) Pub. No.: US 2020/0282188 A1
(43) Pub. Date: Sep. 10, 2020

(54) DRUG LAYER APPLYING DEVICE AND METHOD FOR FORMING DRUG LAYER

(71) Applicant: TERUMO KABUSHIKI KAISHA, Tokyo (JP)

(72) Inventors: Yuno KITAGAWA, Kanagawa (JP); Yasuo KUROSAKI, Kanagawa (JP); Hiroshi GOTO, Kanagawa (JP); Masakazu SHIMOYAMA, Kanagawa (JP)

(73) Assignee: TERUMO KABUSHIKI KAISHA, Tokyo (JP)

(21) Appl. No.: 16/879,277

(22) Filed: May 20, 2020

Related U.S. Application Data
(63) Continuation of application No. PCT/JP2018/043180, filed on Nov. 22, 2018.

(30) Foreign Application Priority Data
Nov. 22, 2017 (JP) 2017-224338

(51) Int. Cl. A61M 25/10; B32B 7/12

(52) U.S. Cl. CPC A61M 25/10; A61M 25/1029; B32B 7/12; B32B 2535/00; A61M 2205/0238; A61M 2025/1031; A61M 2025/105

(57) ABSTRACT

A drug layer applying device and a method for forming a drug layer which can quickly and easily place an appropriate amount of a drug on a surface of a medical instrument. A drug layer applying device that is used by being inserted into a living body and attachable to a balloon, includes: a flexible sheet; and a drug layer provided on one surface of the sheet.

### CROSS-REFERENCES TO RELATED APPLICATIONS

This application is a continuation of International Application No. PCT/JP2018/043180 filed on Nov. 22, 2018, which claims priority to Japanese Application No. 2017-224338 filed on Nov. 22, 2017, the entire content of both of which is incorporated herein by reference.

### FIELD OF THE DISCLOSURE

The present disclosure generally relates to a drug layer applying device that places or applies a drug on a surface of a medical instrument such as a balloon, and a method for forming a drug layer.

### BACKGROUND DISCUSSION

In recent years, a balloon catheter has been used to improve a lesion (stenotic part) generated in a body lumen. The balloon catheter typically includes an elongated shaft portion and a radially inflatable balloon provided on a distal side of the shaft portion. When the deflated balloon is inflated after reaching a target location in a body via a relatively thin body lumen, a lesion can be pushed to be widened.

However, when the lesion is forcibly pushed to be widened, smooth muscle cells may excessively proliferate to cause new stenosis (restenosis) at the lesion. Therefore, recently, a drug eluting balloon (DEB) in which a surface of the balloon is coated with a drug for suppressing stenosis has been used. The drug eluting balloon inflates to instantaneously release the drug with which the surface has been coated to the lesion, thereby suppressing restenosis.

As a method for forming a drug layer on a surface of a balloon, for example, U.S. Pat. No. 8,597,720 B2 describes a method of spraying a solution containing a drug on a balloon, a dipping method, a coating method using a brush, a coating method using a rotating body, and a method of supplying a solution using a pipette.

Japanese Patent Application Publication No. 2010-154919 A describes a device in which a portion between a balloon for dilatation and a balloon for drug supply located outside the balloon for dilatation is filled with a drug. Japanese Patent Application Publication No. 2010-154919 A further describes that a large number of micropores for releasing the drug are formed in the drug supply balloon, and a drug can be additionally supplied from the outside to the portion between the balloon for dilatation and the balloon for drug supply.

With the methods described in U.S. Pat. No. 8,597,720 B2, it can be difficult to quickly apply an appropriate amount of a drug on a surface of a balloon. In addition, the device described in Japanese Patent Application Publication No. 2010-154919 A is capable of supplying the drug to the balloon from the outside, but is incapable of rather easily providing the drug on the balloon due to its complicated structure.

### SUMMARY

A drug layer applying device and a method for forming a drug layer are disclosed, which can relatively quickly and rather easily provide an appropriate amount of a drug on a surface of a medical instrument.

A drug layer applying device is disclosed which is attachable to a medical instrument used by being inserted into a living body and includes: a flexible sheet; and a drug layer provided on one surface of the sheet.

A method is disclosed for forming a drug layer on a surface of a medical instrument insertable into a living body, and includes attaching a surface of a drug layer applying device, provided with a drug layer on one surface of a flexible sheet, to a surface of the medical instrument, the surface of the drug layer applying device opposite to a side where the drug layer of the drug layer applying device is provided.

Another method is disclosed for forming a drug layer which applies a drug on a surface of a balloon, the method comprising: injecting a predetermined amount of an inflation fluid into the balloon; inserting the balloon into a through-hole of a drug layer applying device, the drug layer applying device being a heat-shrinkable tube; and attaching a surface of the drug layer applying device to a surface of the balloon, the surface of the drug layer applying device being opposite to a side of the heat-shrinkable tube to where the drug layer of the drug layer applying device is provided.

The drug layer applying device configured as described above can relatively quickly and rather easily provide the appropriate amount of the drug layer on the surface of the medical instrument by being attached to the surface of the medical instrument.

The drug layer applying device may further include an adhesive layer provided on a surface of the sheet opposite to a side on which the drug layer is provided. As a result, the adhesive layer can adhere to the surface of the medical instrument, and the drug layer can be rather effectively placed on the surface of the medical instrument without being peeled off.

In accordance with an aspect, the sheet may have a cylindrical shape, and the drug layer may be provided on an outer circumferential surface of the sheet. As a result, an appropriate amount of the drug layer can be relatively quickly and rather easily placed on the outer circumferential surface of a cylindrical medical instrument such as a balloon.

The sheet may be a heat-shrinkable tube. As a result, the sheet can be reduced in diameter by heating the medical instrument with the drug layer applying device covered on the medical instrument, and can be placed in contact with the medical instrument.

The adhesive layer may exhibit an adhesive force when heated. As a result, the adhesive layer is also heated when the heat-shrinkable tube is heated, and the adhesive layer exhibits the adhesive force. Therefore, the adhesive layer can be prevented from adhering to an unintended position before the heating. Therefore, the heat-shrinkable tube can be attached to an appropriate position on the surface of the medical instrument after positioning the drug layer with respect to the medical instrument with high precision.

The medical instrument may be a balloon capable of inflating and deflating. As a result, the appropriate amount of the drug layer can be relatively quickly and rather easily placed on the surface of the balloon.

The drug in the drug layer may contain at least one selected from the group including rapamycin, paclitaxel, docetaxel, and everolimus. As a result, restenosis of a stenotic part in a blood vessel can be favorably suppressed by the drug layer.

The drug in the drug layer may contain at least one selected from the group including a water-insoluble drug, a water-soluble drug, and a hydrophilic polymer. As a result, it is possible to apply, to the drug layer, a drug that is appropriate for conditions and the like, alone or in combination, with various other drugs.

With the method for forming a drug layer configured as described above, the appropriate amount of the drug layer can be relatively quickly and rather easily placed on the surface of the medical instrument by attaching the drug layer applying device to the medical instrument.

In the attaching of the surface of the drug layer applying device, providing an adhesive layer provided on a surface of the sheet opposite to the side on which the drug layer may be attached to a surface of the medical instrument. As a result, the adhesive layer can be attached to the surface of the medical instrument, and the drug layer can be rather effectively placed on the surface of the medical instrument without being peeled off.

In the attaching of the surface of the drug layer applying device, the drug layer applying device may be attached to the medical instrument removed from a living body. As a result, the medical instrument that has been used in the living body can be removed from the living body, and then, the drug layer can be placed on the same medical instrument for reuse.

The medical instrument may be a balloon capable of inflating and deflating, guidewire, guiding sheath, guiding catheter, or stent. As a result, it is possible to relatively quickly and rather easily provide an appropriate amount of the drug layer on a surface of the balloon, the guidewire, the guiding sheath, the guiding catheter, or the stent. If the medical instrument is the balloon, the appropriate amount of the drug layer can be relatively quickly and rather easily placed on the surface of the balloon. In addition, the balloon used for pre-dilation of a target site in the living body can be removed, and then, the drug layer can be placed on the same balloon to reuse the balloon for post-dilation of the target site.

### BRIEF DESCRIPTION OF THE DRAWINGS

FIG. 1 is a perspective view illustrating a drug layer applying device according to a first embodiment disclosed here.

FIG. 2 is a cross-sectional view of the drug layer applying device.

FIG. 3 is a front view illustrating a balloon catheter.

FIG. 4 is a front view illustrating a distal portion of the balloon catheter.

FIG. 5 is a cross-sectional view taken along line V-V of FIG. 4.

FIG. 6 is a perspective view illustrating a state where the drug layer applying device is attached to a balloon.

FIG. 7 is a cross-sectional view illustrating the balloon to which the drug layer applying device is attached.

FIG. 8 is a perspective view illustrating a drug layer applying device according to a second embodiment disclosed here.

FIG. 9 is a cross-sectional view taken along line IX-IX of FIG. 8.

FIG. 10 is a front view illustrating a state where a balloon is covered with the drug layer applying device.

FIG. 11 is a cross-sectional view taken along line XI-XI of FIG. 10.

FIG. 12 is a front view illustrating the balloon to which the drug layer applying device is attached.

FIG. 13 is a cross-sectional view taken along line XIII-XIII of FIG. 12.

FIG. 14 is a front view illustrating a balloon to which a drug layer applying device according to a third embodiment is attached.

FIG. 15 is a cross-sectional view illustrating a first modification of the drug layer applying device.

FIGS. 16A and 16B are front views illustrating modifications of the drug layer applying device, in which FIG. 16A illustrates a second modification and FIG. 16B illustrates a third modification.

### DETAILED DESCRIPTION

Set forth below with reference to the accompanying drawings is a detailed description of embodiments of a drug layer applying device that applies a drug on a surface of a medical instrument such as a balloon, and a method for forming a drug layer representing examples of the inventive drug layer applying device that applies a drug on a surface of a medical instrument such as a balloon, and method for forming a drug layer disclosed here. Note that dimensional ratios of the drawings are exaggerated for the convenience of description and may differ from actual ratios in some cases.

### First Embodiment

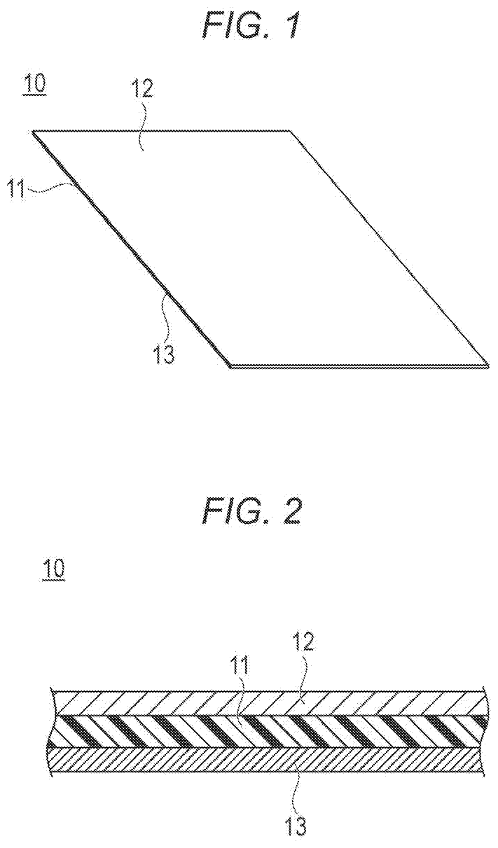

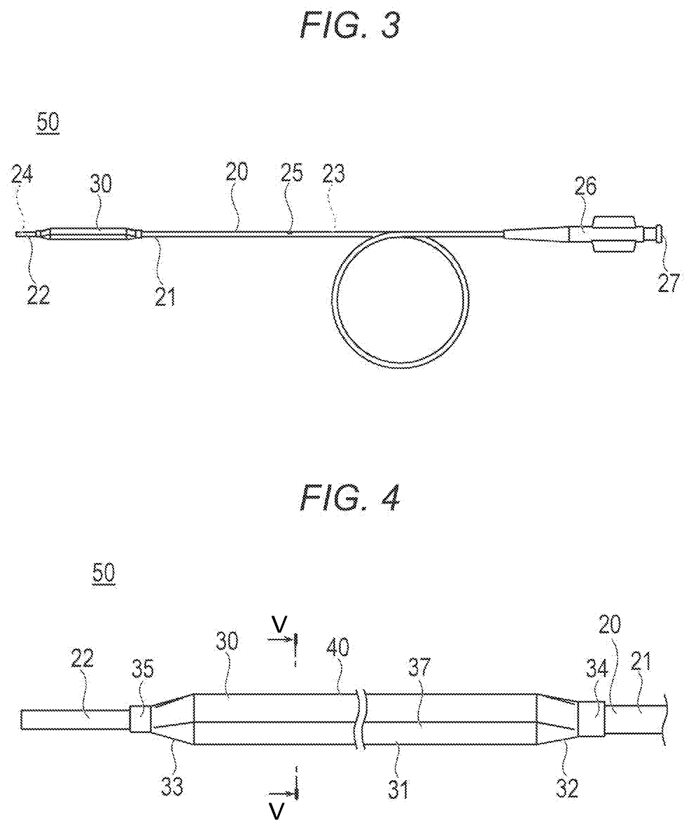

A drug layer applying device (i.e., drug loading instrument) 10 according to a first embodiment of the present disclosure is a device configured to apply a drug layer on a surface of a balloon 30 (see FIG. 3), which is inserted into a stenotic part of a living body lumen, such as a blood vessel, to push and widen the stenotic part, thereby forming a drug-eluting balloon as illustrated in FIGS. 1 and 2. Note that a medical instrument (or medical devices) on which the drug layer applying device 10 applies the drug layer is not limited to the balloon 30, and may be, for example, a guide wire, a guiding sheath, a guiding catheter, a stent, or the like. Hereinafter, a case where the drug layer can be applied to the balloon 30 by the drug layer applying device 10 will be described.

The drug layer applying device 10 can include a flexible sheet 11, a drug layer 12 containing a drug, and an adhesive layer 13 having an adhesive force.

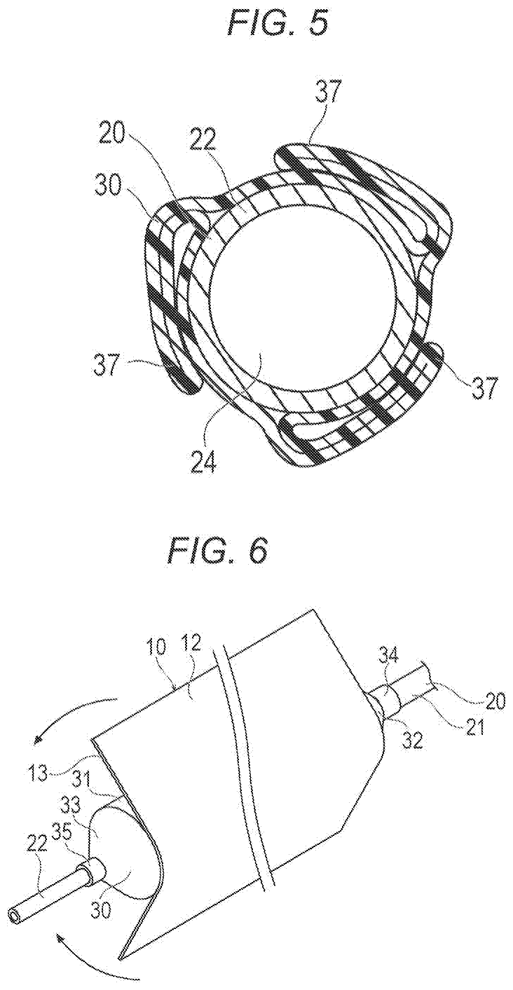

In accordance with an exemplary embodiment, the sheet 11 is a film-shaped member that is flexible and thin. The sheet 11 is preferably relatively thin so as to be foldable together with the balloon 30. A thickness of the sheet 11 can be, for example, 1 μm to 250 μm, preferably 5 μm to 100 μm, and more preferably 10 μm to 50 μm. The sheet 11 may have a concave and/or convex portion shaped to fold the balloon 30. Such shaping can be performed by heating the sheet 11 in the state of being deformed into a predetermined shape by applying a force. The concave and/or convex portion has a function of assisting the drug layer applying device 10 after being attached to the balloon 30 inflated from the folded state (see FIG. 5) to return (rewrap) the balloon 30 to the folded state.

The material of the sheet 11 can include, for example, polyolefin, polyvinyl chloride, polystyrene, polyethylene, polypropylene, polyethylene terephthalate, a fluoropolymer, a thermoplastic elastomer, a nonwoven fabric, and the like.

The drug layer 12 is provided on one surface of the sheet 11. The drug contained in the drug layer 12 may be a water-soluble drug or a water-insoluble drug. A water-insoluble drug means a drug that is insoluble or poorly soluble in water, and specifically solubility in water may be less than 1 mg/mL, and further, may be less than 0.1 mg/mL. Water-insoluble drugs can include fat-soluble drugs. An amount of the drug contained in the drug layer 12 is not particularly limited, but the drug is contained at a density, for example, of 0.1 μg/mm² to 10 μg/mm², preferably at a density of 0.5 μg/mm² to 5 μg/mm², and more preferably at a density of 0.5 μg/mm² to 3.5 μg/mm². A thickness of the drug layer 12 is not particularly limited, but can be, for example, 0.1 μm to 100 μm, preferably 0.5 μm to 50 μm, and more preferably 0.5 μm to 10 μm or 10 μm to 30 μm. A form of the water-insoluble or water-soluble drug is not particularly limited, and may be, for example, a crystal or not.

The water-insoluble drug can include, for example, immunosuppressants, for example, cyclosporines containing cyclosporine, immunoadjuvants such as rapamycin, carcinostatics such as paclitaxel, antiviral agents or antibacterial agents, antineoplastic agents, analgesic agents and anti-inflammatory agents, antibiotics, antiepileptics, anxiolytic agents, antiparalytic agents, antagonists, neuron blocking agents, anticholinergic agents and cholinergic agents, muscarine antagonists agents and muscarine agents, antiadrenergic agents, antiarrhythmic agents, antihypertensive agents, hormone preparations, and nutritional supplements.

The water-insoluble drug is preferably, for example, at least one selected from the group including rapamycin, paclitaxel, docetaxel, and everolimus. The rapamycin, paclitaxel, docetaxel, and everolimus in the present specification can include their analogs and/or derivatives as long as the analogs and/or derivatives have equivalent drug effect. For example, paclitaxel and docetaxel are in an analog relation. Rapamycin and everolimus are in a derivative relation among these, paclitaxel is more preferable.

The water-soluble drug may be a drug having solubility in water of 1 mg/mL or more, preferably 5 mg/mL or more, more preferably 10 mg/mL or more, and still more preferably 33 mg/mL or more. Water-soluble antiplatelet drugs can include, for example, clopidogrel sulfate, ticlopidine hydrochloride, prasugrel hydrochloride, sarpogrelate hydrochloride, and the like (incidentally, water-insoluble antiplatelet drugs include aspirin, cilostazol, ticagrelor, and the like). Examples of the water-soluble anticoagulant can include warfarin, edoxaban tosilate hydrate, heparin, dabigatran etexilate methanesulfonate, and the like. The drug may also be a hydrophilic polymer, and a wet coating using the hydrophilic polymer (the coating that exhibits lubricity when wetted with water) is possible. The drug may be applied as the hydrophilic polymer. on a surface (inner and outer surfaces) of a medical instrument to be inserted into a blood vessel (for example, a guidewire, a guiding sheath, a guiding catheter, or the like) without being limited to the surface (inner and outer surfaces) of the balloon catheter.

The drug layer 12 may contain an additive (for example, an excipient). When the drug layer 12 contains the additive, examples of the additive can include, for example, a water-soluble low molecular weight compound and the like. A molecular weight of the water-soluble low molecular weight compound is 50 to 2,000, preferably 50 to 1,000, more preferably 50 to 500, and still more preferably 50 to 200. An amount of the water-soluble low molecular weight compound is preferably 10 parts by weight to 5,000 parts by weight, more preferably 50 parts by weight to 3000 parts by weight, and still more preferably 100 parts by weight to 1000 parts by weight, per 100 parts by weight of the water-insoluble drug. The water-soluble low molecular weight compound material can be, for example, a serine ethyl ester, sugars such as glucose, sugar alcohols such as sorbitol, citrate, polysorbate, polyethylene glycol, and urea.

In accordance with an embodiment, the water-soluble low molecular weight compound can be water-soluble polymers, a contrast agent, an amino acid ester, a glycerol ester of a short-chain monocarboxylic acid, a pharmaceutically acceptable salt, and a surfactant, or a mixture of two or more of the water-soluble low molecular weight compound materials can be used. The water-soluble low molecular weight compound has a hydrophilic group and a hydrophobic group, and can be characterized by being soluble in water. The water-soluble low molecular weight compound is preferably non-swellable or hardly swellable (i.e., only a small part of a compound is swellable). The additive containing the water-soluble low molecular weight compound has an effect of uniformly dispersing the water-insoluble drug on the surface of the sheet 11. In accordance with an embodiment, it can be preferable that the additive is not a hydrogel. The additive contains the low molecular weight compound, and thus, dissolves relatively quickly without swelling when coming into contact with an aqueous solution. Further, the additive can rather easily dissolve when the balloon 30 is inflated in the blood vessel so that crystal particles of the water-insoluble drug on the surface of the balloon 30 are rather easily released, and thus, there is an effect of increasing the number of the crystal particles of the drug adhering to the blood vessel.

The water-soluble low molecular weight compound has a molecular weight of 50 to 2,000, and can be dissolved at an amount of 1 mg/mL or more in water, preferably dissolved at an amount of 5 mg/mL or more in water, more preferably dissolved at an amount of 10 mg/mL or more in water, still more preferably dissolved at an amount of 33 mg/mL or more in water, and preferably dissolved in water without inflating. In accordance with an exemplary embodiment, it can be preferable that the water-soluble low molecular weight compound is not a hydrogel. The water-soluble low molecular weight compound is preferably not a polymer, and more preferably not a water-insoluble polymer. In accordance with an exemplary embodiment, it can be preferable that the water-soluble low molecular weight compound is not polyethylene glycol (PEG) and a water-soluble PEG (for example, polyethylene glycol 200-600).

The solubility of a substance can be defined as a degree of dissolution within 30 minutes at 20° C. For example, the solubility of a substance can be defined by an amount of solvent (for example, an amount of water) required to dissolve 1 g (or 1 mL) of solute. When the amount of solvent required to dissolve 1 g of solute is less than 1 mL, the solute is extremely soluble in the solvent. In cases, of extremely soluble, the amount of dissolved solute is more than 1000 mg/mL. Examples of extremely soluble substances can include sorbitol, urea, and glycerol. When the amount of solvent required to dissolve 1 g of solute is 1 mL or more and less than 10 mL, the solute is freely soluble in the solvent. In cases of freely soluble, the amount of dissolved solute may be more than 100 mg/mL and 1000 mg/mL or less. Examples of freely soluble substances can include polysorbate, an amino acid ester, polyethylene glycol 200-600, a serine ethyl ester, a contrast agent (iopromide), and a water-soluble polymer. When the amount of solvent required to dissolve 1 g of solute is 10 mL or more and less than 30 mL, the solute is soluble in the solvent. In cases of soluble, the amount of dissolved solute may be more than 33 mg/mL and 100 mg/mL or less. Examples of soluble substances can include polyethylene glycol. When the amount of solvent required to dissolve 1 g of solute is 30 mL or more and less than 100 mL, the solute is slightly soluble in the solvent. In case of slightly soluble, the amount of dissolved solute may be more than 10 mg/mL and 33 mg/mL or less. When the amount of solvent required to dissolve 1 g of solute is 100 mL or more and less than 1000 mL, the solute is sparingly soluble in the solvent. In cases of sparingly soluble, the amount of dissolved solute may be more than 1 mg/mL and 10 mg/mL or less. When the amount of solvent required to dissolve 1 g of solute is 1000 mL or more and less than 10,000 mL, the solute is extremely insoluble in the solvent. In cases of extremely insoluble, the amount of dissolved solute may be more than 0.1 mg/mL and 1 mg/mL or less. When the amount of solvent required to dissolve 1 g of solute is 10,000 mL or more, the solute is hardly soluble in the solvent. In cases of hardly soluble, the amount of dissolved solute may be 0.1 mg/mL or less. Examples of hardly soluble substances can include a fatty acid ester of glycerin. The water-soluble substance refers to a substance other than a substance that is “extremely insoluble” and a substance that is “hardly soluble”. Specifically, the water-soluble substance indicates a substance that is “extremely soluble”, a substance that is “freely soluble”, a substance that is “slightly soluble”, and a substance that is “sparingly soluble”. The water-soluble substance preferably indicates a substance that is “extremely soluble”, a substance that is “freely soluble” and a substance that is “slightly soluble”.

In accordance with an exemplary embodiment, the adhesive layer 13 is provided on a surface of the sheet 11 opposite to the side on which the drug layer 12 is provided. The adhesive layer 13 is a layer adhering to the surface of the balloon 30. A thickness of the adhesive layer 13 is not particularly limited, for example, the thickness of the adhesive layer 13 can be 0.01 μm to 50 μm, preferably 0.1 μm to 30 μm, and more preferably 0.1 μm to 5 μm.

The material of the adhesive layer 13 may be water-soluble or water-insoluble. Examples of the water-soluble adhesive can include a vinyl chloride resin adhesive, a vinyl acetate copolymer resin adhesive, an EVA resin adhesive, an acrylic resin adhesive, an acrylic ester adhesives, a styrene/butadiene copolymer latex, an aqueous urethane adhesive, and the like. The adhesive layer 13 can be, for example, a pressure-sensitive adhesive that adheres by pressing. Examples of the pressure-sensitive adhesive can include a natural rubber latex adhesive, a silicone pressure-sensitive adhesive, an MG latex adhesive, an acrylic adhesive, a silica adhesive, and the like.

The drug layer applying device 10 preferably has a size that can cover a range where a drug of the balloon 30 is applied (or placed). For example, when the drug is placed on a straight portion 31 of the balloon 30, the drug layer applying device 10 preferably has a size that can cover the straight portion 31.

Next, a balloon catheter 50 on which a drug is applied using the drug layer applying device 10 will be described with reference to FIGS. 3 to 5. In the present specification, a side of the balloon catheter 50 to be inserted into a living body lumen is referred to as a “distal side” and an operating hand side is referred to as a “proximal side”.

The balloon catheter 50 can include an elongated shaft portion 20, the balloon 30 provided at a distal portion of the shaft portion 20, and a hub 26 fixed to a proximal end of the shaft portion 20.

The shaft portion 20 can include an outer tube 21 that is a tubular body of which distal end and proximal end are open and an inner tube 22 which is a tubular body provided inside the outer tube 21. The inner tube 22 is housed in a hollow interior of the outer tube 21, and the shaft portion 20 has a double-tube structure at the distal portion. The hollow interior of the inner tube 22 is a guide wire lumen 24 through which a guide wire is inserted. An inflation lumen 23 for circulating inflation fluid of the balloon 30 is formed in the hollow interior of the outer tube 21 outside the inner tube 22. The inner tube 22 is open to the outside at a side opening 25. The inner tube 22 protrudes further to the distal side from the distal end of the outer tube 21. A distal tip, which is a separate member, may be provided at a distal portion of the inner tube 22.

The balloon 30 (medical instrument) can include a straight portion 31 formed at the center in the axial direction, a proximal tapered portion 32 located on the proximal side of the straight portion 31, and a distal tapered portion 33 located on the distal side of the straight portion 31. The straight portion 31 can have a cylindrical shape that has substantially the same outer diameter when inflated. An outer diameter of the proximal tapered portion 32 gradually decreases from the straight portion 31 toward the proximal side. An outer diameter of the distal tapered portion 33 gradually decreases from the straight portion 31 toward the distal side.

In accordance with an exemplary embodiment, the straight portion 31 is a portion where the drug is applied by the drug layer applying device 10. Note that the range in which the drug is applied by the drug layer applying device 10 is not limited only to the straight portion 31, but may include at least a part of the proximal tapered portion 32 and the distal tapered portion 33 in addition to the straight portion 31. Alternatively, the range in which the drug is applied by the drug layer applying device 10 may be only a part (or portion) of the straight portion 31.

In the balloon 30, a balloon fusing portion 34 located at the proximal end of the proximal tapered portion 32 can be fused at the distal portion of the outer tube 21. In addition, a balloon fusing portion 35 located at the distal end of the distal tapered portion 33 can be fused to the distal portion of the inner tube 22 in the balloon 30. Note that a method for fixing the balloon 30 to the outer tube 21 and the inner tube 22 is not limited to the fusion, but may be, for example, adhesion. As a result, the interior of the balloon 30 communicates with the inflation lumen 23. The balloon 30 can be inflated by injecting the inflation fluid into the balloon 30 via the inflation lumen 23. In accordance with an embodiment, the inflation fluid may be a gas or a liquid, and, for example, a gas such as a helium gas, a CO₂ gas, an O₂ gas, an N₂ gas, an Ar gas, air, and a mixed gas, or a liquid such as a saline solution and a contrast agent can be used.

In accordance with an embodiment, the balloon 30 can have a plurality of blade portions 37 shaped to protrude in the radial direction. The blade portions 37 can be folded in the circumferential direction. The blade portion 37 is formed by a fold extending substantially in the axial direction of the balloon 30. In accordance with an embodiment, a length of the blade portion 37 in the long-axis direction (i.e., axial direction) does not exceed a length of the balloon 30. The number of the blade portions 37 is not particularly limited, and can be one to seven, for example, however, as shown in the present embodiment, the number of blades portions 37 is three. The plurality of blade portions 37 are preferably arranged to be uniform in the circumferential direction of the balloon 30, but are not limited to being arranged to be uniform in the circumferential direction of the balloon 30.

The length of the balloon 30 in the axial direction is not particularly limited, but is preferably, for example, 5 mm to 500 mm, more preferably 10 mm to 300 mm, and still more preferably 20 mm to 200 mm. The outer diameter of the balloon 30 when inflated is not particularly limited, but can be, for example, 1 mm to 10 mm, and more preferably 2 mm to 8 mm.

It is preferable that the balloon 30 have a certain degree of flexibility and a certain degree of hardness such that the balloon 30 can be inflated when reaching a blood vessel, a tissue, or the like, and release the drug on the surface of the balloon 30. Specifically, the balloon 30 can be made of metal or resin. At least the surface of the balloon 30 is preferably made of resin. The material of at least the surface of the balloon 30, can be, for example, polyolefins such as polyethylene, polypropylene, polybutene, an ethylene-propylene copolymer, an ethylene-vinyl acetate copolymer, and an ionomer, or a mixture of two or more of the polyolefins, thermoplastic resins such as soft polyvinyl chloride resin, polyamide, a polyamide elastomer, a nylon elastomer, polyester, a polyester elastomer, polyurethane, and a fluororesin, a silicone rubber, a latex rubber, and the like can be used. In accordance with an exemplary embodiment, the material of the at least the surface of the balloon 30, polyamides are preferably used.

In the hub 26, a proximal opening 27 is formed, which communicates with the inflation lumen 23 of the outer tube 21 and functions as a port for inflow and outflow of the inflation fluid.

Next, an operation of the drug layer applying device 10 according to the present embodiment will be described.

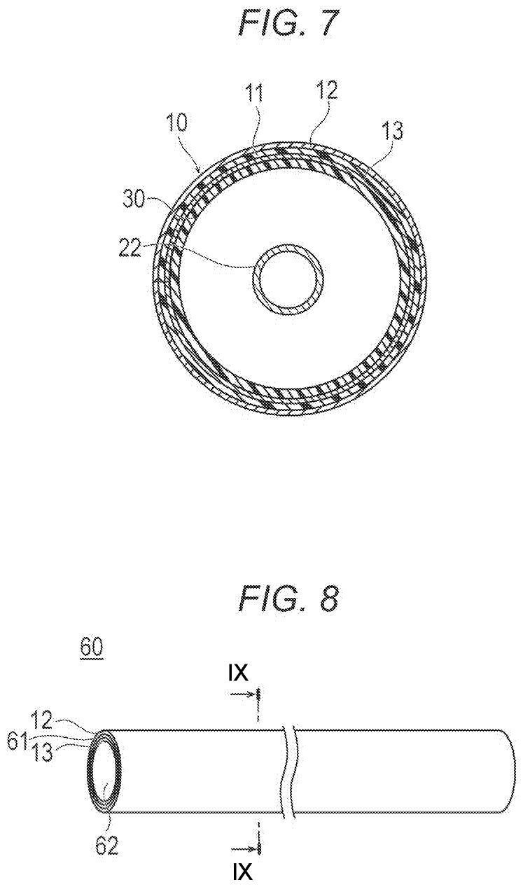

When applying the drug on the balloon 30 by the drug layer applying device 10, a predetermined amount of the inflation fluid is injected from the proximal opening 27 of the hub 26 using an indeflator, a syringe, or the like to send the inflation fluid inside the balloon 30 through the inflation lumen 23. As a result, the folded balloon 30 inflates. Next, the adhesive layer 13 of the drug layer applying device 10 is pressed against the straight portion 31 of the inflated balloon 30 as illustrated in FIGS. 6 and 7. As a result, the adhesive layer 13 is attached to the straight portion 31. When the drug layer applying device 10 is larger than the straight portion 31, the drug layer applying device 10 may be cut into an appropriate size.

Next, the inflation fluid is suctioned (i.e., sucked or removed) and discharged from the interior of the balloon 30 through the proximal opening 27 of the hub 26. As a result, the balloon 30 is deflated and folded. As a result, the balloon 30 can be used for expansion of a stenotic part in a living body lumen, such as a blood vessel, as a drug eluting balloon.

As described above, the drug layer applying device 10 according to the present embodiment is the drug layer applying device 10 that is attachable to the balloon 30 (medical instrument) used by being inserted into the living body, and can include the flexible sheet 11 and the drug layer 12 provided on one surface of the sheet 11.

The drug layer applying device 10 configured as described above can relatively quickly and rather easily place the appropriate amount of the drug layer 12 on the surface of the balloon 30 by being attached to the surface of the balloon 30. The drug layer applying device 10 can relatively quickly and rather easily place the drug layer 12 on the balloon 30, and thus, can be also used in the state of covering the balloon 30 after use, for example, in a clinical field regardless of a place of use. Therefore, for example, the drug layer applying device 10 can be applied to the balloon 30 removed from the living body after being used for pre-dilation of the stenotic part to obtain the balloon 30 for post-dilation having the drug layer 12. Therefore, when a balloon for pre-dilation and a balloon for post-dilation are required, the single balloon 30 can fulfill the two roles. In addition, it is also possible to appropriately select and use an appropriate drug layer applying device 10, for example, from among a plurality of drug layer applying devices 10 having different sizes, types of drugs, amounts of drugs, and the like. In addition, the drug layer applying device 10 can be provided in the form of the sheet, and thus, a large drug layer applying device 10 can be cut out to an appropriate size in accordance with a diameter and a length of the balloon 30 and used, for example, in a clinical field.

The drug layer applying device 10 further includes the adhesive layer 13 provided on the surface of the sheet 11 opposite to the side on which the drug layer 12 is provided. As a result, the adhesive layer 13 can adhere to the surface of the balloon 30, and the appropriate amount of the drug layer 12 can be effectively applied to the surface of the balloon 30 without being peeled off.

In addition, the adhesive layer 13 may be water-soluble. As a result, the adhesive layer 13 can exhibit a favorable adhesiveness since the balloon 30 contains moisture. Therefore, the surface of the balloon 30 may be wetted before the balloon 30 is covered with the drug layer applying device 10. In addition, the balloon 30 removed from the living body after the pre-dilation is highly likely to contain moisture, and the adhesiveness can be improved.

In accordance with an aspect, the water-insoluble drug in the drug layer 12 contains at least one selected from the group including rapamycin, paclitaxel, docetaxel, and everolimus. As a result, restenosis of the stenotic part in the blood vessel can be favorably suppressed by the drug layer 12.

In accordance with another aspect, the drug in the drug layer 12 may contain at least one selected from the group including a water-insoluble drug, a water-soluble drug, and a hydrophilic polymer. As a result, it is possible to apply, to the drug layer 12, a drug that is appropriate for conditions and the like, alone or in combination with various other drugs.

The medical instrument to which the drug layer applying device 10 is attached is the balloon 30 that is capable of inflating and deflating. Therefore, the drug layer 12 can be relatively quickly and rather easily placed on the surface of the balloon 30.

In addition, the present disclosure also includes a method for forming a drug layer configured to apply a drug on the surface of the balloon 30. The method for forming the drug layer which applies the drug on the surface of the balloon 30 (medical instrument) used by being inserted into the living body, and includes: attaching the surface of the drug layer applying device 10, provided with the drug layer 12 on one surface of the flexible sheet 11, to the surface of the balloon 30, the surface of the drug layer applying device 10 opposite to the side where the drug layer 12 of the drug layer applying device 10 is provided.

According to the method for forming a drug layer configured as described above, an appropriate amount of the drug can be relatively quickly and rather easily placed on the surface of the balloon 30 by attaching the drug layer applying device 10 to the balloon 30.

In the attaching of the surface of the drug layer applying device 10 to the surface of the balloon 30, the adhesive layer 13 provided on the surface of the sheet 11 opposite to the side where the drug layer 12 is provided may be attached to the surface of the balloon 30. As a result, the adhesive layer 13 can adhere to the surface of the balloon 30, and the drug layer 12 can be effectively applied to the surface of the balloon 30 without being peeled off.

In the attaching of the surface of the drug layer applying device 10 to the surface of the balloon 30, the drug layer applying device 10 may be attached to the balloon 30 removed from the inside of the living body. As a result, the balloon 30 that has been used in the living body can be removed from the living body, and then, the drug layer 12 can be placed on the same balloon 30 for reuse.

The medical instrument to which the drug layer applying device 10 is attached is the balloon 30 that is capable of inflating and deflating. As a result, the appropriate amount of the drug layer 12 can be relatively quickly and rather easily placed on the surface of the balloon 30. In addition, the balloon 30 that has been used for pre-dilation of a target site of the living body can be removed, and then, the drug layer 12 can be placed on the same balloon 30 to reuse the balloon 30 for post-dilation of the target site.

### Second Embodiment

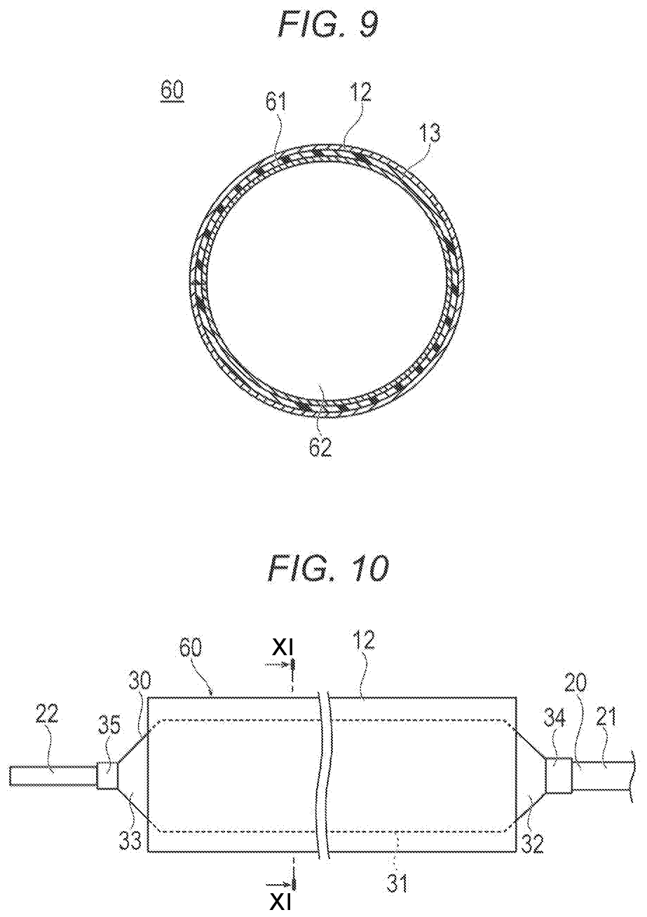

A drug layer applying device 60 according to a second embodiment of the present disclosure is different from the first embodiment in terms of being cylindrical as illustrated in FIGS. 8 and 9. Note that parts having the same functions as those in the first embodiment are denoted by the same reference signs, and the description of those parts having the same functions as those in the first embodiment will be omitted.

In accordance with an embodiment, the drug layer applying device 60 can include: a heat-shrinkable tube 61 (sheet) that shrinks when heated; the drug layer 12 containing a drug; and the adhesive layer 13 having an adhesive force. The drug layer 12 is provided on an outer circumferential surface of the heat-shrinkable tube 61. The adhesive layer 13 is provided on an inner circumferential surface of the heat-shrinkable tube 61.

The heat-shrinkable tube 61 as the sheet is a tube (i.e., tubular member) of which diameter is reduced when heated. The heat-shrinkable tube 61 has strength enough to maintain a through-hole 62. Note that the heat-shrinkable tube 61 may be a cylindrical film. The cylindrical film can be flexible and thin, and thus, does not always have such strength as to be capable of maintaining the through-hole 62 and can be deformed into a flat plate shape such that the through-hole 62 is closed.

The material of the heat-shrinkable tube 61 is not limited as long as the material of the heat-shrinkable tube 61 can be reduced in diameter by heating, and is preferably a material capable of coating the inner circumferential surface with a drug. In accordance with an embodiment, the heat-shrinkable tube 61 preferably shrinks at a relatively low heating temperature. The temperature at which the heat-shrinkable tube 61 shrinks can be, for example, 40° C. to 150° C., preferably 40° C. to 100° C. Since the heat-shrinkable tube 61 shrinks at a relatively low temperature, deterioration of the drug, deformation of the balloon 30, and the like can be suppressed. A shrinkage ratio of an inner diameter of the heat-shrinkable tube 61 (inner diameter after shrinkage/inner diameter before shrinkage) is not particularly limited, but is preferably 40% to 80%. The heat-shrinkable tube 61 may have a concave and/or convex portion shaped to fold the balloon 30. Such shaping of the concave and/or convex portion can be performed by heating the sheet 11 in the state of being deformed into a predetermined shape by applying a force. The concave and/or convex portion has a function of assisting the drug layer applying device 60 after being attached to the inflated balloon 30 to return (rewrap) the balloon 30 to the folded state.

The material of the tubular heat-shrinkable tube 61 can include, for example, polyolefin, a fluorine-based polymer, polyvinyl chloride, thermoplastic elastomer, and the like.

The material of the heat-shrinkable tube 61 in the case of a tubular film can be, for example, polyolefin, polyvinyl chloride, polystyrene, polyethylene, polypropylene, polyethylene terephthalate, or the like.

The adhesive layer 13 may be a material that exhibits an adhesive force by raising its temperature to a temperature at which the heat-shrinkable tube 61 is heated. Examples of the material of the adhesive layer 13 that exhibits the adhesive force when heated can include a styrene-butadiene rubber-based adhesive, a poly (lactide-co-glycotide) copolymer, a polymer such as polycaprolactone, a surfactant such as polyethylene glycol, a polyoxyethylene fatty acid diester, a polyoxyethylene fatty acid monoester, and a polyoxyethylene polyoxypropylene block polymer, an α-cyanoacrylate adhesive, and a fibrin adhesive used as medical adhesives, and the like.

The drug layer applying device 60 is used by housing the balloon 30 in an inflated state in the drug layer applying device 60. Therefore, an inner diameter of the drug layer applying device 60 is preferably equal to or larger than an outer diameter of the inflated balloon 30.

It is preferable that an axial length of the drug layer applying device 60 be equal to or longer than an axial length of a range in which the drug of the balloon 30 is applied when the drug layer applying device 60 is heated to shrink. In the present embodiment, the axial length of the drug layer applying device 60 exceeds a length of the straight portion 31.

Next, an operation of the drug layer applying device 60 according to the second embodiment will be described.

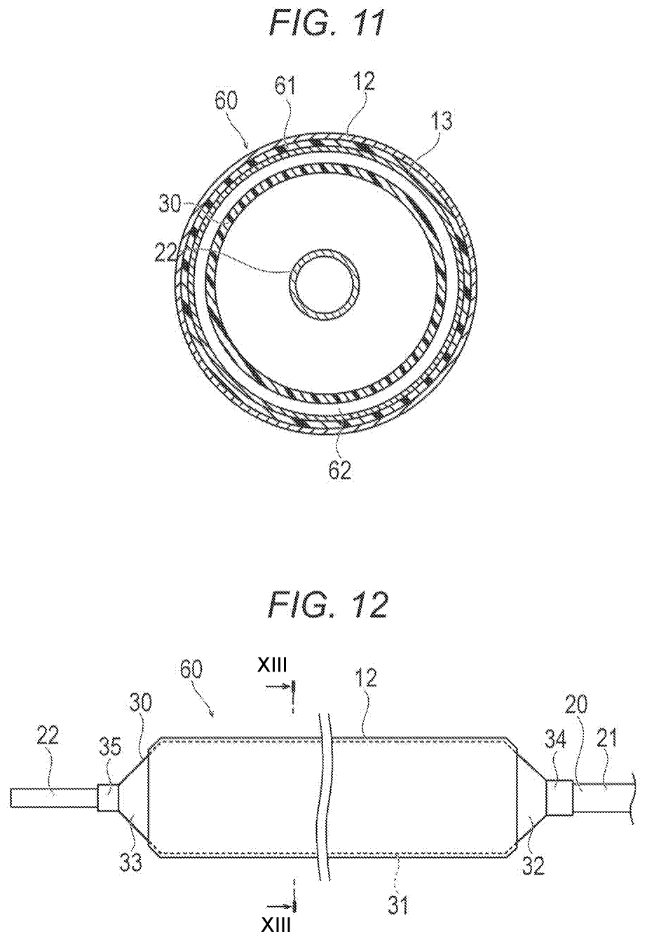

When applying the drug on the balloon 30 by the drug layer applying device 60, a predetermined amount of the inflation fluid is injected from the proximal opening 27 of the hub 26 using an indeflator (i.e., an inflation/deflation device), a syringe, or the like to send the inflation fluid inside the balloon 30 through the inflation lumen 23. As a result, the folded balloon 30 inflates as illustrated in FIGS. 10 and 11. Next, the balloon 30 is inserted into the through-hole 62 of the drug layer applying device 60. Note that the balloon 30 may be inflated after inserting the balloon 30 into the through-hole 62.

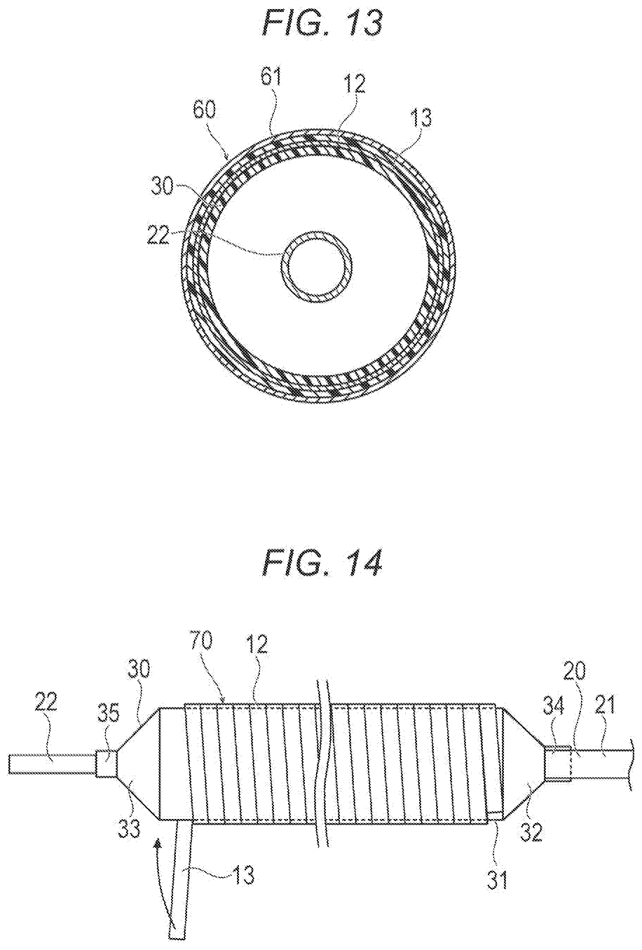

Next, the drug layer applying device 60 is heated to a temperature at which the heat-shrinkable tube 61 shrinks by a dryer, an oven, or the like that supplies hot air when a current flows. As a result, the heat-shrinkable tube 61 can be reduced in diameter, and the adhesive layer 13 is in contact with the balloon 30 as illustrated in FIGS. 12 and 13. As a result, the adhesive layer 13 adheres to the surface of the balloon 30. When the adhesive layer 13 is an adhesive that exhibits an adhesive force by heating, the adhesive layer 13 is also heated when the heat-shrinkable tube 61 is heated, and the adhesive layer 13 adheres to the surface of the balloon 30. If an axial length of the drug layer applying device 60 with the heat shrunken heat-shrinkable tube 61 applied to the drug layer applying device 60 exceeds the length of the straight portion 31, the drug layer applying device 60 with the heat shrunken heat-shrinkable tube 61 applied to the drug layer applying device 60 can cover the entire straight portion 31. Further, a distal end of the shrunk drug layer applying device 60 covers a proximal portion of the distal tapered portion 33, and a proximal end of the shrunk drug layer applying device 60 can cover a distal portion of the proximal tapered portion 32. As a result, both the ends of the drug layer applying device 60 are smaller in diameter than the portion that covers the straight portion 31 and thermally shrink. As a result, the drug layer applying device 60 is firmly fixed to the balloon 30 and is not detachable. Note that the distal end of the shrunk drug layer applying device 60 does not necessarily cover the proximal portion of the distal tapered portion 33. In addition, the proximal end of the shrunk drug layer applying device 60 do not necessarily cover the distal portion of the proximal tapered portion 32.

Next, the inflation fluid is suctioned (i.e., sucked) and discharged from the interior of the balloon 30 through the proximal opening 27 of the hub 26. As a result, the balloon 30 is deflated and folded. As a result, the balloon 30 can be used for expansion of a stenotic part in a living body lumen, such as a blood vessel, as a drug eluting balloon.

As described above, the sheet according to the second embodiment is cylindrical, and the drug layer 12 is provided on the outer circumferential surface of the sheet. As a result, the appropriate amount of the drug layer 12 can be relatively quickly and rather easily placed on the outer circumferential surface of the cylindrical medical instrument such as the balloon 30.

In addition, the sheet of the drug layer applying device 60 is the heat-shrinkable tube 61. As a result, when the balloon 30 covered with the drug layer applying device 60 is heated, the heat-shrinkable tube 61 as the sheet can be reduced in diameter, and the balloon 30 can be brought into contact with the balloon.

The adhesive layer 13 may exert an adhesive force when heated. As a result, the adhesive layer 13 is also heated when the heat-shrinkable tube 61 is heated, and the adhesive layer 13 exhibits the adhesive force. Therefore, it is possible to prevent the adhesive layer 13 from adhering to an unintended position before the heating. Therefore, the heat-shrinkable tube 61 can be attached to an appropriate position on the surface of the balloon 30 after positioning the drug layer 12 with respect to the balloon 30 with high precision.

### Third Embodiment

A drug layer applying device 70 according to a third embodiment of the present disclosure is different from the first embodiment in terms of being a strip-shaped long tape as illustrated in FIG. 14. Note that parts having the same functions as those in the first embodiment are denoted by the same reference signs, and the description of those parts having the same function as those in the first embodiment will be omitted. A structure (the sheet 11, the drug layer 12, and the adhesive layer 13) of the drug layer applying device 70 other than the shape is the same as that of the first embodiment.

When using the drug layer applying device 70, the drug layer applying device 70 is cut into a length in accordance with a size of the balloon 30, and the resultant is spirally wound around the inflated balloon 30 to attach the adhesive layer 13 to the balloon 30. As a result, the drug layer 12 having an appropriate size can be rather easily placed or applied on the balloon 30 having desired size.

Note that the present disclosure is not limited to only the above-described embodiment, and various modifications can be made by those skilled in the art within a technical idea of the present disclosure. For example, the balloon catheter 50 can be a rapid exchange type, but may be an over-the-wire type.

In addition, a target to which the drug layer 12 is applied by the drug layer applying device 10, 60, or 70 is not limited to the balloon 30 as long as the target is a medical instrument that is used by being inserted into a living body, and may be, for example, a stent, a covered stent, an implant, or the like.

In addition, the drug layer applying device is not necessarily provided with the adhesive layer as long as the drug layer applying device is attached to the surface of the balloon 30. In this case, an adhesive is applied to an adhering surface of the drug layer applying device or the surface of the balloon 30 at the time of attaching the drug layer applying device to the balloon 30. The adhesive is not particularly limited, but is preferably a liquid adhesive, and examples of the liquid adhesive can include a cyanoacrylate-based instant adhesive, a fibrin adhesive, a starch-based adhesive, a natural rubber-based adhesive, a cellulose-based adhesive, a polyimide-based adhesive, and the like. As a result, the drug layer applying device can be attached to the surface of the balloon 30 even if the drug layer applying device is not provided with an adhesive layer in advance.

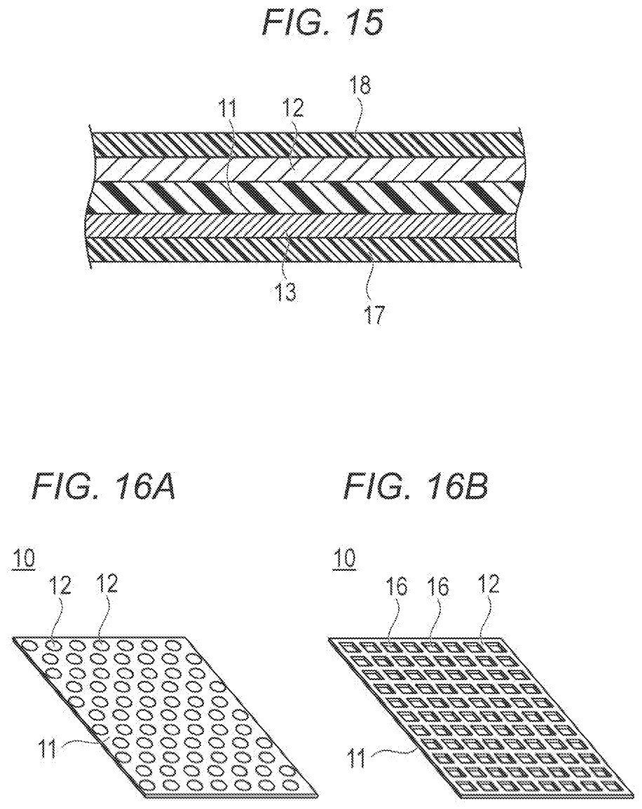

In addition, a protective film 17 that can be peeled off from the adhesive layer 13 may be attached to the adhesive layer 13 as in a first modification illustrated in FIG. 15. As a result, it is possible, for example, to suppress dust and the like from adhering to the adhesive layer 13 before use. The protective film 17 can be rather easily peeled off before attaching the adhesive layer 13 to the balloon 30. In addition, a drug protective film 18 covering the drug layer 12 may be attached to the drug layer 12. The drug protective film 18 is attached to the drug layer 12 in a peelable manner. Alternatively, the drug protective film 18 may be a water-soluble film. In this case, the balloon 30 can be inserted into a blood vessel without removing the drug protective film 18 from the drug layer 12 after attaching the adhesive layer 13 to the balloon 30. The balloon catheter 50 to which the drug layer applying device equipped with the drug protective film 18 is attached can suppress detachment of the drug when inserted into the blood vessel. The material of the drug protective film 18 can include, for example, a gelatin film, a collagen film, a starch film, and the like. The drug protective film 18 may be attached to the drug layer 12 via a biocompatible adhesive. Examples of the biocompatible adhesive can include a cyanoacrylate-based adhesive, a gelatin-based adhesive, a fibrin-based adhesive, and the like.

In addition, the drug layer 12 may be partially placed on the surface of the sheet 11 (or the heat-shrinkable tube 61) as in a second modification illustrated in FIG. 16A. Note that a shape of the range where the drug layer 12 is provided is not particularly limited. Therefore, the range in which the drug layer 12 is provided can be set to a desired range in the drug layer applying device 10, 60, or 70.

In addition, the drug layer applying device 10, 60, or 70 may have a plurality of holes 16 as in a third modification illustrated in FIG. 16B. The number and shapes of the holes 16 are not particularly limited.

The detailed description above describes embodiments of a drug layer applying device that applies a drug on a surface of a medical instrument such as a balloon, and a method for forming a drug layer. The invention is not limited, however, to the precise embodiments and variations described. Various changes, modifications and equivalents may occur to one skilled in the art without departing from the spirit and scope of the invention as defined in the accompanying claims. It is expressly intended that all such changes, modifications

What is claimed is:

1. A drug layer applying device which is attachable to a medical instrument used by being inserted into a living body, the drug layer applying device comprising:
a flexible sheet; and
a drug layer provided on one surface of the sheet.

2. The drug layer applying device according to claim 1, further comprising:
an adhesive layer provided on a surface of the sheet opposite to a side on which the drug layer is provided.

3. The drug layer applying device according to claim 1, wherein the sheet has a cylindrical shape, and the drug layer is provided on an outer circumferential surface of the sheet.

4. The drug layer applying device according to claim 1, wherein the sheet is a heat-shrinkable tube.

5. The drug layer applying device according to claim 1, wherein the adhesive layer exhibits an adhesive force when heated.

6. The drug layer applying device according to claim 1, wherein the medical instrument is a balloon that is configured to inflate and deflate.

7. The drug layer applying device according to claim 1, wherein the drug in the drug layer contains at least one selected from a group consisting of a water-insoluble drug, a water-soluble drug, and a hydrophilic polymer.

8. The drug layer applying device according to claim 1, wherein the flexible sheet is a strip-shaped long tape that is spirally wound around the medical instrument.

9. A method for forming a drug layer which applies a drug on a surface of a medical instrument insertable into a living body, the method comprising
attaching a surface of a drug layer applying device, provided with the drug layer on one surface of a flexible sheet, to a surface of the medical instrument, the surface of the drug layer applying device being attached to the surface being opposite to a side where the drug layer of the drug layer applying device is provided.

10. The method for forming a drug layer according to claim 9, wherein in the attaching of the surface of the drug layer applying device to the surface of the medical instrument, the method comprises:
providing an adhesive layer on a surface of the sheet opposite to the side where the drug layer is attached to the surface of the medical instrument.

11. The method for forming a drug layer according to claim 9, wherein in the attaching of the surface of the drug layer applying device to the surface of the medical instrument, the method comprises:
attaching the drug layer applying device to the medical instrument removed from an inside of the living body.

12. The method for forming a drug layer according to claim 9, wherein the medical instrument is a balloon, a guidewire, a guiding sheath, a guiding catheter, or a stent which is capable of inflating and deflating.

13. A method for forming a drug layer which applies a drug on a surface of a balloon, the method comprising:
injecting a predetermined amount of an inflation fluid into the balloon;
inserting the balloon into a through-hole of a drug layer applying device, the drug layer applying device being a heat-shrinkable tube; and
attaching a surface of the drug layer applying device to a surface of the balloon, the surface of the drug layer applying device being opposite to a side of the heat-shrinkable tube to where the drug layer of the drug layer applying device is provided.

14. The method for forming a drug layer according to claim 13, further comprising:
further inflating the balloon after the balloon has been inserted into the through-hole of the drug layer applying device.

15. The method for forming a drug layer according to claim 13, wherein the surface of the drug layer applying device that has been attached to the surface of the balloon includes an adhesive layer, the method further comprising:
heating the drug layer applying device to a temperature at which the heat-shrinkable tube shrinks to adhere the drug layer applying device to the surface of the balloon.

16. The method for forming a drug layer according to claim 15, further comprising:
removing the inflation fluid from an interior of the balloon after the heat-shrinkable tube shrinks to adhere the drug layer applying device to the surface of the balloon.

17. The method for forming a drug layer according to claim 13, wherein the balloon include a straight portion formed at a center in the axial direction, a proximal tapered portion located on a proximal side of the straight portion, and a distal tapered portion located on the distal side of the straight portion, the method comprising:
attaching the drug layer applying device to only the straight portion of the balloon.
